(19) United States
(12) Patent Application Publication
(10) Pub. No.: US 2012/0035480 A1
(43) Pub. Date: Feb. 9, 2012

(54) ULTRASONIC DIAGNOSTIC DEVICE

(75) Inventors: Manabu Migita, Kanagawa (JP); Kazuya Takagi, Osaka (JP)

(73) Assignee: PANASONIC CORPORATION, Osaka (JP)

(21) Appl. No.: 13/263,597

(22) PCT Filed: Aug. 9, 2010
(86) PCT No.: PCT/JP2010/005005

§ 371 Date: Oct. 7, 2011

(30) Foreign Application Priority Data
Aug. 18, 2009 (JP) 2009-189431

(51) Int. Cl. A61B 8/14

(52) U.S. Cl. 600/443

(57) ABSTRACT

An ultrasonic diagnostic apparatus according to the present invention sends out an ultrasonic wave toward a subject and receives the ultrasonic wave reflected from the subject, thereby generating the subject's tomographic image. The apparatus includes: a receiving section 214 for generating a number of received signals, which belong to the same acoustic line but which have mutually different reception sensitivities, based on a detection signal that has been obtained by detecting the ultrasonic reflected wave that has returned from the subject; a synthesis processing section 206 for classifying segments on the acoustic line according to the intensity of the ultrasonic reflected wave, thereby making a synthetic received signal out of the received signals based on a result of the classification; and an image generating section 207 for generating the subject's tomographic image by using either grayscales or color tones based on the signal intensity of the synthetic received signal.

### TECHNICAL FIELD

The present invention relates to an ultrasonic diagnostic apparatus and more particularly relates to an ultrasonic diagnostic apparatus for displaying a tomographic image.

### BACKGROUND ART

An ultrasonic diagnostic apparatus sends out an ultrasonic wave toward a subject and analyzes the information included in its reflected echo, thereby generating an image representing an internal body issue of the subject. In this case, various kinds of information can be obtained by numerous methods for sending out the ultrasonic wave or analyzing the reflected echo. Among other things, a so-called “B-mode” method for displaying an image representing the structure of a subject's internal body tissue or a so-called “color flow mode” method for displaying an image representing the blood flow is generally used. However, as these two methods require mutually different ultrasonic wave driving methods, it is usually difficult to display those images by the two methods at the same time.

To overcome such a problem, another display method called “B-flow mode” with the features of those two methods in combination has been developed recently (see Patent Document No. 1 and Non-Patent Document No. 1). That B-flow mode is characterized in that the sensitivity of reception is increased in order to display an image of blood flow that has no strong reflector. That is why the B-flow mode could be said to be a sort of B-mode with high reception sensitivity.

FIG. 16 is a block diagram illustrating an example of a conventional ultrasonic diagnostic apparatus that conducts a B-mode display operation. As shown in FIG. 16, the conventional ultrasonic diagnostic apparatus includes a transmitting section 110, a receiving section 111, a delay adding section 103, a detecting section 104, a logarithm compressing section 105, a scan converting section 106, an attenuation correction control section 107 and a transmission and reception control section 108.

Under the control by the transmission and reception control section 108, the transmitting section 110 outputs a signal to send to a probe 109. In response, the probe 109 sends out an ultrasonic wave toward a subject. Next, the ultrasonic wave that has been reflected from the subject is detected as a reflected echo by the probe 109. As a result, the detected signal is input to the receiving section 111.

The receiving section 111 includes an amplifying section 101 and an A/D converting section 102. The amplifying section 101 amplifies the detected signal, thereby generating a received signal. In this case, the degree of amplification is determined by the amplification factor specified by the attenuation correction control section 107. The deeper the level the reflected echo has come from, the more significantly the echo will have been attenuated. That is why to make the intensity of a reflected echo that has come from a shallow level in the subject and that of another reflected echo that has come from a deep level in the subject apparently equal to each other, the attenuation correction control section 107 increases the amplification factor as the time passes since the signal was received. Then, the A/D converting section 102 converts the received signal thus generated into a digital signal.

The delay adding section 103 performs a focus control on the digital received signal. The detecting section 104 performs envelope detection on the received signal that has been subjected to the focus control. Next, the logarithm compressing section 105 subjects the received signal detected to a logarithm compression in order to compress the dynamic range. Then, a scan converting section 106 generates display image data based on the received signal that has been subjected to the logarithm compression. And the display section 112 displays the image data thus generated.

### CITATION LIST

### Patent Literature

Patent Document No. 1: Japanese Patent Application Laid-Open Publication No. 2004-129967

### Non-Patent Literature

Non-Patent Document No. 1: GE Healthcare, Ultrasonic Diagnostic Apparatus, [online], [searched the Web on Jul. 14, 2009], <URL:http://japan.gehealthcare.com/cwcjapan/static/rad/us/msujbflw.html/>
Non-Patent Document No. 2: Wikipedia, [online], [searched the Web on Jul. 14, 2009], <http://en.wikipedia.org/wiki/High dynamic segment imaging>

### SUMMARY OF INVENTION

### Technical Problem

FIGS. 17 and 18 schematically illustrate images representing a blood vessel that was captured by an ultrasonic diagnostic apparatus that carries out a conventional B-mode display method. Specifically, in FIG. 17, the reception sensitivity is set to be relatively low. That is why a blood flow region 150 where there is no strong reflector is displayed in solid black, thus preventing the user from sensing any blood flow. On the other hand, in FIG. 18, the reception sensitivity is set to be relatively high. In that case, the blood flow region 150 certainly has increased visibility but the vascular wall 151, which is a strong reflector, has caused overexposure. Thus, even if the reception sensitivity setting is simply increased in such an ultrasonic diagnostic apparatus that conducts the conventional B-mode display, not both of the blood flow region and the body tissue region such as the vascular wall cannot be presented as an image in appropriate grayscales. In other words, the B-flow mode cannot be realized.

According to Non-Patent Document No. 1, the reception sensitivity is increased by a known code modulation method called “coded excitation”, thereby realizing the B-flow mode. Nevertheless, if the receiving section does not have a sufficiently broad dynamic range, overexpose could still arise in a region where there is a strong reflector.

It is therefore an object of the present invention to provide method for generating an ultrasonic image without causing underexposure in any dark region or overexposure in any bright region even if the receiving section does not have a sufficiently broad dynamic range.

### Solution to Problem

An ultrasonic diagnostic apparatus according to the present invention sends out an ultrasonic wave toward a subject and receives the ultrasonic wave that has been reflected from the subject, thereby generating the subject's tomographic image. The apparatus includes: a receiving section for generating a number of received signals, which belong to the same acoustic line but which have mutually different reception sensitivities, based on a detection signal that has been obtained by detecting the ultrasonic reflected wave that has returned from the subject; a synthesis processing section for classifying segments on the acoustic line according to the intensity of the ultrasonic reflected wave, thereby making a synthetic received signal out of the received signals based on a result of the classification; and an image generating section for generating the subject's tomographic image by using either grayscales or color tones based on the signal intensity of the synthetic received signal. Thus, even if the receiving section does not have a sufficiently broad dynamic range, the tomographic image can be displayed in appropriate grayscales or color tones without causing underexposure in any dark region or overexposure in any bright region.

In one preferred embodiment, the received signals with the different reception sensitivities include a received signal with higher reception sensitivity and a received signal with lower reception sensitivity. The synthesis processing section generates the synthetic received signal by applying the received signal with the higher reception sensitivity to a segment on the acoustic line where the ultrasonic wave has been reflected relatively faintly and the received signal with the lower reception sensitivity to a segment on the acoustic line where the ultrasonic wave has been reflected relatively strongly.

In this particular preferred embodiment, the receiving section amplifies the detection signal with multiple different amplification factors, thereby generating the received signal with the higher reception sensitivity and the received signal with the lower reception sensitivity. In this manner, a number of signals with different reception sensitivities can be obtained.

In another preferred embodiment, the ultrasonic diagnostic apparatus further includes a transmitting section for generating a pulse signal and a coded pulse signal and driving a probe that sends out the ultrasonic wave. The receiving section generates the received signal with the higher reception sensitivity by making the probe detect and demodulate the ultrasonic wave that has been sent out as the coded pulse signal and also generates the received signal with the lower reception sensitivity by making the probe receive the ultrasonic wave that has been sent out as the pulse signal. Thus, a signal with high reception sensitivity can be obtained.

In still another preferred embodiment, the ultrasonic diagnostic apparatus further includes a transmitting section for driving a probe that sends out the ultrasonic wave. The transmitting section drives and instructs the probe to scan the subject with ultrasonic waves that have been sent out n times (where n is an integer that is equal to or greater than two) per acoustic line. And the receiving section amplifies the detection signal that has been obtained by the probe with the higher amplification factor and with the lower amplification factor alternately. In this manner, a number of signals with mutually different reception sensitivities can be obtained time-sequentially. As a result, a signal with a relatively broad dynamic range can be generated with the received signal with the higher reception sensitivity and the received signal with the lower reception sensitivity used in combination.

In yet another preferred embodiment, the receiving section amplifies the ultrasonic wave detection signal with a higher amplification factor and with a lower amplification factor in parallel with each other, thereby generating the received signal with the higher reception sensitivity and the received signal with the lower reception sensitivity. In this manner, two received signals with mutually different reception sensitivities can be obtained without decreasing the frame rate.

In yet another preferred embodiment, the receiving section amplifies the detection signal with the amplification factor increased with time while the ultrasonic wave is being transmitted with respect to the same acoustic line. As a result, amplification for the purpose of attenuation correction and amplification for the purpose of expanding the dynamic range can be carried out simultaneously.

In yet another preferred embodiment, based on a result of statistical analysis on the signal intensities of the detection signal, the synthesis processing section automatically determines a segment with the lower signal intensity and a segment with the higher signal intensity. As a result, the decision can be made without depending on the sensitivity unique to any particular apparatus or system.

### Advantageous Effects of Invention

According to the present invention, multiple received signals with mutually different reception sensitivities are synthesized together, and a tomographic image is generated in either grayscales or color tones corresponding to the signal intensities by using such a synthetic received signal. As a result, a good tomographic image can be displayed in either grayscales or color tones over the entire segment including not only dark regions but also bright regions as well.

### BRIEF DESCRIPTION OF DRAWINGS

FIG. 1 illustrates conceptually how to process received signals by high dynamic range imaging.

FIG. 2 is a block diagram illustrating an ultrasonic diagnostic apparatus as a first preferred embodiment of the present invention.

FIG. 3 is a flowchart illustrating how the ultrasonic diagnostic apparatus shown in FIG. 2 works.

FIG. 4 schematically shows a sequence in which the ultrasonic diagnostic apparatus shown in FIG. 2 sends out ultrasonic waves.

FIG. 5 schematically shows a sequence in which the ultrasonic diagnostic apparatus shown in FIG. 2 controls the amplification factors.

FIG. 6 schematically shows another sequence in which the ultrasonic diagnostic apparatus shown in FIG. 2 controls the amplification factors.

FIG. 7 schematically shows another sequence in which the ultrasonic diagnostic apparatus shown in FIG. 2 sends out ultrasonic waves.

FIG. 8 is a block diagram illustrating a configuration for the synthesis processing section of the ultrasonic diagnostic apparatus shown in FIG. 2.

FIG. 9 is a flowchart showing how the synthesis processing section operates.

FIG. 10 is a schematic representation illustrating an exemplary tomographic image generated by the ultrasonic diagnostic apparatus shown in FIG. 2.

FIG. 11 is a block diagram illustrating an ultrasonic diagnostic apparatus as a second preferred embodiment of the present invention.

FIG. 12 is a flowchart illustrating how the ultrasonic diagnostic apparatus shown in FIG. 11 works.

FIG. 13 is a block diagram illustrating an ultrasonic diagnostic apparatus as a third preferred embodiment of the present invention.

FIG. 14 is a flowchart illustrating how the ultrasonic diagnostic apparatus shown in FIG. 13 works.

FIG. 15 illustrates an example in which a synthetic received signal is generated from three received signals with mutually different reception sensitivities.

FIG. 16 is a block diagram illustrating a conventional ultrasonic diagnostic apparatus.

FIG. 17 is a schematic representation illustrating a tomographic image generated by a conventional ultrasonic diagnostic apparatus.

FIG. 18 is a schematic representation illustrating another tomographic image generated by a conventional ultrasonic diagnostic apparatus.

### DESCRIPTION OF EMBODIMENTS

To avoid causing underexposure in a dark region of a tomographic image being displayed in grayscales or overexposure in a bright region thereof, an ultrasonic diagnostic apparatus according to the present invention generates a subject's tomographic image by high dynamic range imaging. By adopting the high dynamic range imaging method, even if the receiving section of the ultrasonic diagnostic apparatus has a narrow dynamic range, a good tomographic image can be displayed in appropriate grayscales over the entire range including not only dark regions but also bright regions as well.

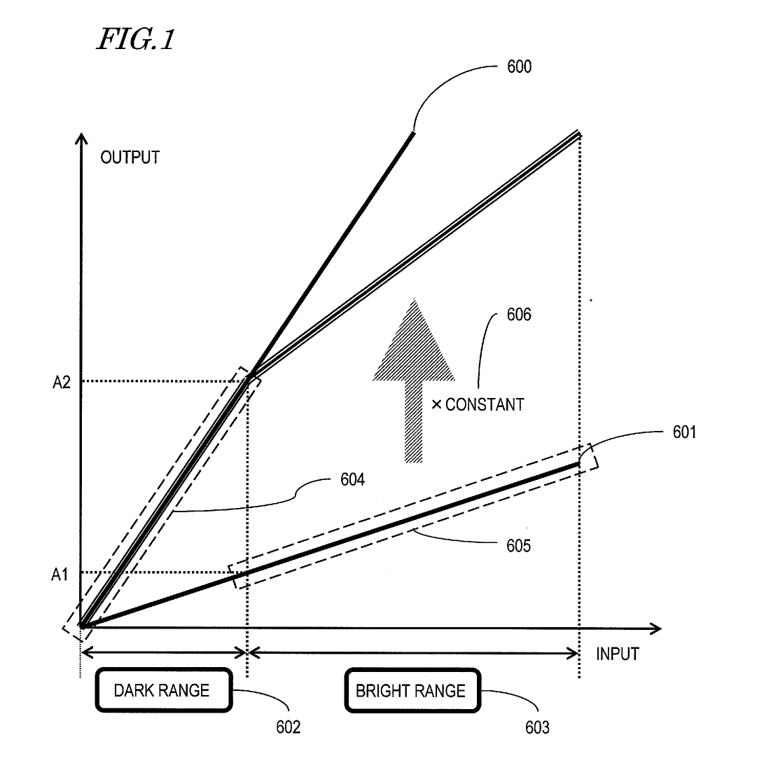

FIG. 1 schematically illustrates the concept of the high dynamic range imaging. In FIG. 1, the abscissa represents the intensity of a detection signal, which is obtained by getting a reflected echo that has returned from the subject detected by a probe. On the other hand, the ordinate represents the intensity of a received signal, which is obtained by getting the detection signal amplified by a receiving section.

As shown along the axis of abscissas, a segment (range) in which the detection signal has a relatively low intensity corresponds to a dark region of a tomographic image generated and has a faint reflected echo, while a segment (range) in which the detection signal has a relatively high intensity corresponds to a bright region of the tomographic image generated and has a strong reflected echo. In FIG. 1, those segments are shown as a dark segment 602 and a bright segment 603, respectively.

According to the present invention, a received signal with higher reception sensitivity and a received signal with lower reception sensitivity are generated from a detection signal that has been obtained with the same acoustic line. In FIG. 1, shown as an example are a received signal 601 with the lower reception sensitivity, which has been obtained by amplifying the detection signal with a small amplification factor, and a received signal 600 with the higher reception sensitivity, which has been obtained by amplifying the detection signal with a large amplification factor.

The dark segment 602 corresponds to a segment where there is no strong reflector (such as blood flow). And a portion 604 of the received signal 600 with the higher reception sensitivity is used in this segment 602. By applying the received signal 600 with the higher reception sensitivity to the dark segment 602, grayscales can be increased in the dark segment, and therefore, the underexposure can be avoided.

On the other hand, the bright segment 603 corresponds to a segment where there is a strong reflector (such as vascular wall). And a portion 605 of the received signal 601 with the lower reception sensitivity is used in this segment 603. As a result, overexposure that would be caused by too high reception sensitivity can be reduced significantly in this bright segment 603.

As can be seen from FIG. 1, when respective portions of the two received signals in those two segments are synthesized together, there is a significant intensity level difference between the received signals in the boundary between the dark and bright segments 602 and 603 because those two received signals are associated with mutually different amplification factors. Thus, to iron out the difference, the portion 605 of the received signal 601 with the lower reception sensitivity is multiplied by a constant 606, thereby making the portion 604 of the received signal 600 with the higher reception sensitivity continuous with the portion 605 of the received signal 601 with the lower reception sensitivity. Instead of multiplying that portion 605 by the constant 606, a constant may be added to it.

As also can be seen from FIG. 1, the dark and bright segments 602 and 603 can be defined on each acoustic line by either classifying or determining those segments on the acoustic line according to either the intensity of the detection signal or the signal intensity of the received signal 601 with the lower reception sensitivity or the received signal 600 with the higher reception sensitivity. The dark and bright segments 602 and 603 can be defined by adopting a threshold value A1 when the received signal 600 with the higher reception sensitivity is used and by adopting a threshold value A2 when the received signal 601 with the lower reception sensitivity is used, respectively.

As described above, by synthesizing together those received signals with two different reception sensitivities and by generating a tomographic image in grayscales corresponding to the signal intensities using that synthetic received signal, a good tomographic image can be generated in appropriate grayscales over the entire region including dark and bright regions. In the preferred embodiments to be described below, two received signals with mutually different reception sensitivities are supposed to be synthesized together. However, three or more received signals with different reception sensitivities may be used as well.

### Embodiment 1

Hereinafter, a first preferred embodiment of an ultrasonic diagnostic apparatus according to the present invention will be described.

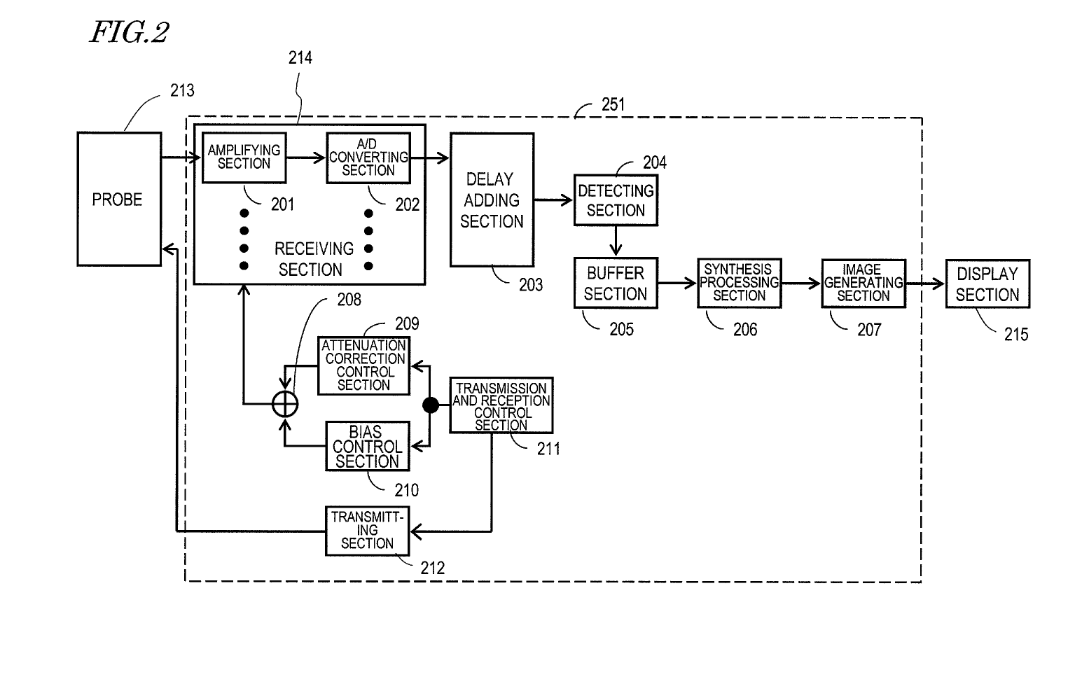

FIG. 2 is a block diagram illustrating a configuration for an ultrasonic diagnostic apparatus 251, which includes a transmitting section 212, a transmission and reception control section 211, a bias control section 210, an attenuation correction control section 209, a receiving section 214, a delay adding section 203, a detecting section 204, a buffer section 205, a synthesis processing section 206 and an image generating section 207. Also, a probe 213 for transmitting and receiving an ultrasonic wave is connected to the transmitting section 212 and the receiving section 214. And a display section 215 for displaying the image generated is connected to the image generating section 207. In this case, the probe 213 and the display section 215 may form integral parts of this ultrasonic diagnostic apparatus 215 or may be general-purpose ones. The probe 213 includes a number of vibrators, which are arranged one dimensionally and which may be made of a piezoelectric material, for example. That is to say, an ultrasonic wave is transmitted by driving the piezoelectric vibrators, and is received and transformed into an electrical signal by the piezoelectric vibrators.

Under the control of the transmission and reception control section 211, the transmitting section 212 outputs a signal to send to the probe 213. In response, the probe 213 is driven and transmits an ultrasonic wave toward a subject. The ultrasonic wave is then reflected from the subject and detected as a reflected echo by the probe 213. As a result, a detection signal is input to the receiving section 214. According to this preferred embodiment, to obtain two received signals with mutually different reception sensitivities (specifically, a received signal with higher reception sensitivity and a received signal with lower reception sensitivity) on the same acoustic line, ultrasonic waves are transmitted and received twice on the same acoustic line.

The receiving section 214 includes amplifying sections 201 and A/D converting sections 202. In this case, the number of amplifying sections 201 and A/D converting sections 202 provided is preferably the same as that of the vibrators included in the probe 213. Each of the amplifying sections 201 amplifies the detection signal that has been obtained by an associated one of the vibrators in the probe 213, thereby generating a received signal. In this case, the amplification factors provided by the attenuation control section 209 and the bias control section 210 are added together by an adder 208 at the timing specified by the transmission and reception control section 211 and the detection signal is amplified with the amplification factor thus calculated. To obtain two received signals with mutually different reception sensitivities, the adder 208 outputs two different amplification factors at two different times that are controlled by the transmission and reception control section 211. Each of the A/D converting sections 202 converts the received signal thus obtained into a digital signal. Next, the delay adding section 203 performs a focus control on the digital received signal. Then, the detecting section 204 carries out an envelope detection on the focus controlled received signal, and a detected received signal gets stored in the buffer section 205. As a result, data about the received signal with the higher reception sensitivity and the received signal with the lower reception sensitivity are collected.

The synthesis processing section 206 retrieves the data from the buffer section 205, and generates a synthetic received signal on a frame-by-frame basis by the high dynamic range imaging method so that the received signal with the higher reception sensitivity is applied to a segment with a the fainter reflected echo and the received signal with the lower reception sensitivity is applied to a segment with the stronger reflected echo on the same acoustic line as already described with reference to FIG. 1. Then, the image generating section 207 generates the subject's tomographic image in either grayscales or color tones corresponding to the signal intensity of the synthetic received signal. As a result, the subject's tomographic image is presented on the display section 215.

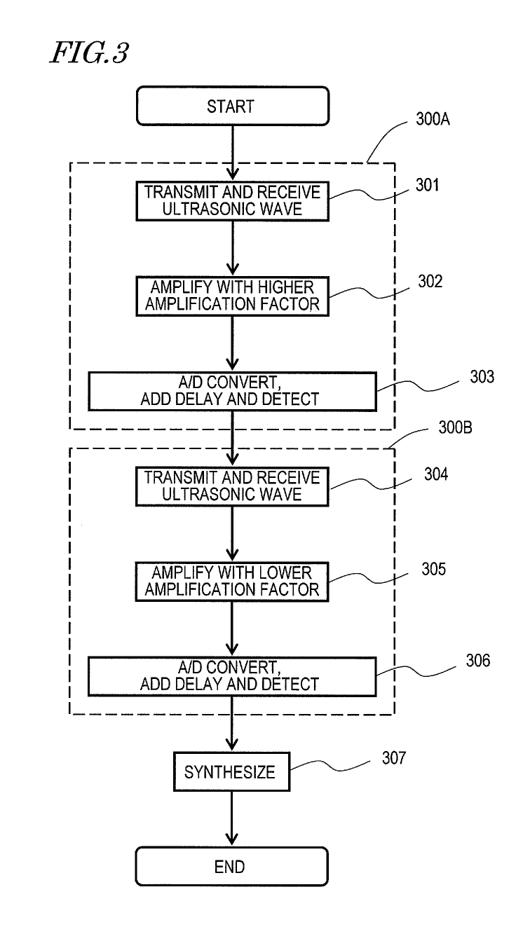

Hereinafter, it will be described in detail with reference to FIGS. 2 and 3 how this ultrasonic diagnostic apparatus 251 operates. FIG. 3 is a flowchart illustrating how this ultrasonic diagnostic apparatus 251 works. As shown in FIG. 3, after having started the measuring process, the ultrasonic diagnostic apparatus 251 performs two series of steps 300A and 300B. In this case, each of these two series of steps is carried out on the same acoustic line when an ultrasonic wave is transmitted and received once. As a result, a received signal with higher reception sensitivity and a received signal with lower reception sensitivity can be obtained on the same acoustic line.

<Step 301>

An ultrasonic wave is transmitted and received for the first time. For that purpose, the probe 213 is driven by the transmitting section 212 to send out an ultrasonic wave toward the subject and then detects a reflected echo and generates a detection signal.

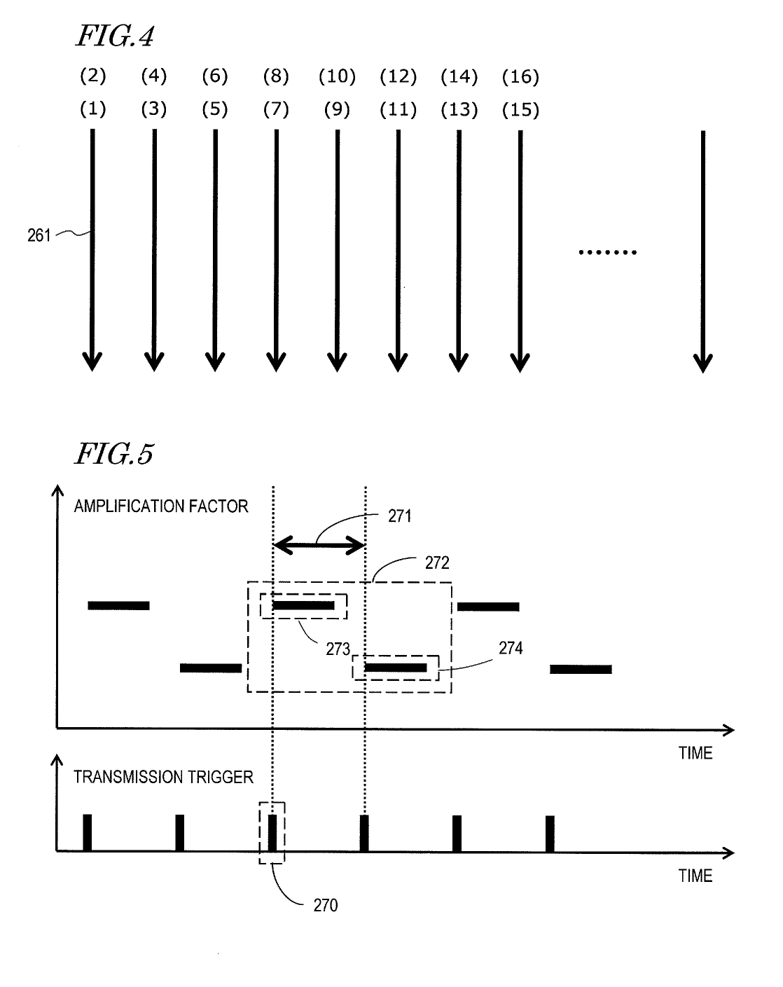

FIG. 4 schematically indicates the sequence in which ultrasonic waves are transmitted from the probe 213. As shown in FIG. 4, after ultrasonic waves have been transmitted twice with respect to the same acoustic line 261, ultrasonic waves are transmitted twice again on the adjacent acoustic line. By sending out two ultrasonic waves per acoustic line in this manner, the probe 213 is driven so as to scan the subject.

In this case, the first transmission and reception of the ultrasonic wave on the same acoustic line 261 is included in the series of steps 300A, and the second transmission and reception of the ultrasonic wave on the same acoustic line 261 is included in the series of steps 300B.

<Step 302>

Each detection signal is amplified by its associated amplifying section 201, thereby generating a received signal. The amplification factor for the received signal is provided by the adder 208. When the timing to send out an ultrasonic wave is specified by the transmission and reception control section 211, the bias control section 210 is triggered and keeps the amplification factor to output to the adder 208 constant until the next transmission of an ultrasonic wave is instructed. In this case, according to the ultrasonic waves to be transmitted twice on the same acoustic line, either the received signal with the higher reception sensitivity or the received signal with the lower reception sensitivity may be generated earlier than the other.

FIG. 5 shows an exemplary amplification factor control sequence. The transmission trigger 270 is the timing to send out an ultrasonic wave as specified by the transmission and reception control section 211. In each period 271 that is defined by the transmission interval between two transmission triggers 270, an ultrasonic wave is sent out and a received signal is generated. In the amplification factor control sequence shown in FIG. 5, the amplification factor 273 is constant. In this case, even a detection signal representing a reflected echo that has come from a deep level in the subject is also amplified with the same amplification factor. In order to generate a received signal with the higher reception sensitivity, the amplification factor 273 is set to be high.

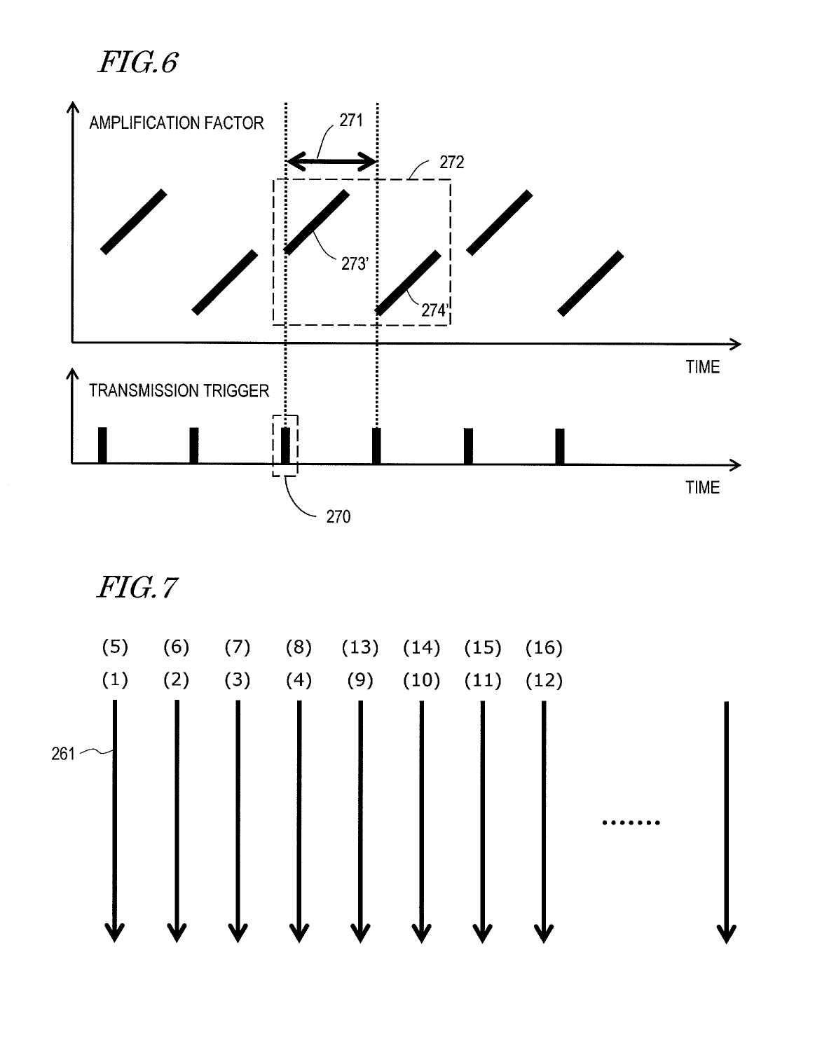

FIG. 6 shows another amplification factor control sequence. In the example illustrated in FIG. 6, at the timing of the transmission trigger 270 as specified by the transmission and reception control section 211, the attenuation correction control section 209 outputs a signal that gradually increases the amplification factor while a reflected echo is being received to the adder 208. In response, the adder 208 sets the sum of the amplification factors that have been provided by the attenuation correction control section 209 and the bias control section 210 with respect to the amplifying section 201. As a result, in the period 271, the amplification factor 273′ increases with time as shown in FIG. 6.

<Step 303>

Each A/D converting section 202 converts the received signal that has been amplified by its associated amplifying section 201 into a digital signal. Next, the delay adding section 203 performs a focus control on the digital received signal. Then, the detecting section 204 carries out an envelope detection on the focus controlled received signal, and a detected received signal gets stored in the buffer section 205.

<Step 304>

An ultrasonic wave is transmitted and received for the second time. For that purpose, the probe 213 is driven by the transmitting section 212 to send out an ultrasonic wave toward the subject and then detects a reflected echo and generates a detection signal.

<Step 305>

As in the processing step 302, each detection signal is amplified by its associated amplifying section 201, thereby generating a received signal. When an ultrasonic wave is transmitted and received for the second time, however, the amplification factor 274 is set to be low as shown in FIG. 5 in order to generate a received signal with the lower reception sensitivity. The transmission and reception of ultrasonic waves on a single acoustic line is completed in the period 272 in which the amplification factors 273 and 274 are output. If the attenuation correction control section 209 outputs a signal that gradually increases the amplification factor while the reflected echo is being received to the adder 208, then the amplification factor provided by the bias control section 210 is decreased. As a result, the adder 208 outputs an amplification factor 274′, which is smaller than the amplification factor 273′ and which is used to amplify the detection signal for the second time. In this manner, a received signal is generated for the second time.

<Step 306>

As in the processing step 303, the received signal is converted into a digital signal, and subjected to a focus control and then to envelope detection by the detecting section 204. As a result, a detected received signal gets stored in the buffer section 205.

<Step 307>

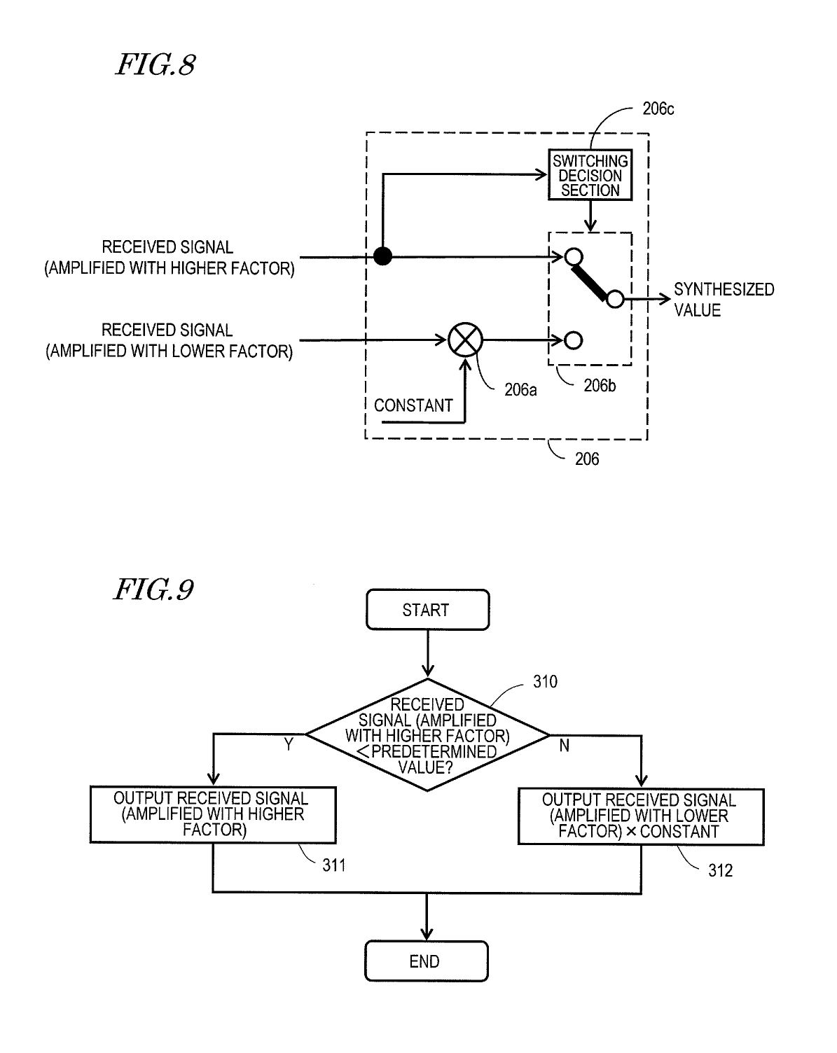

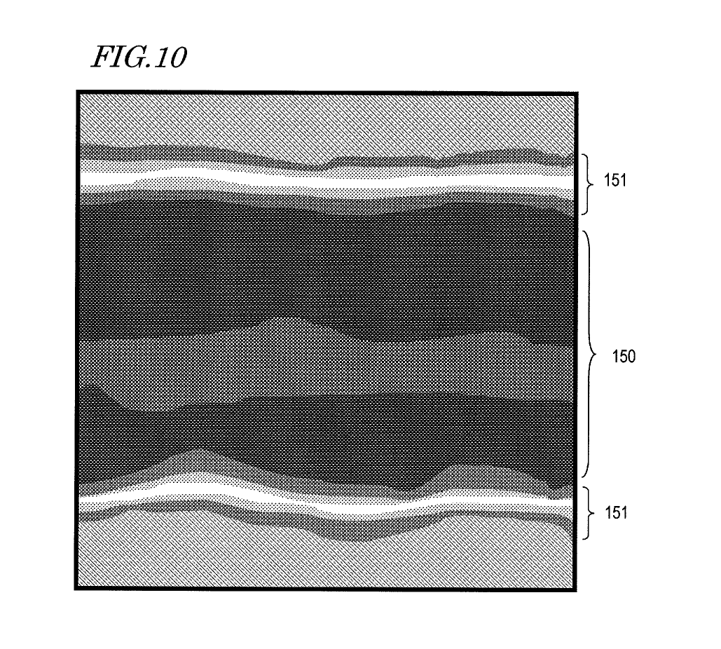

The synthesis processing section 206 synthesizes together the first and second received signals that respectively have higher and lower reception sensitivities and that have been stored, thereby generating a synthetic received signal. FIG. 8 is a block diagram illustrating a configuration for the synthesis processing section 206.

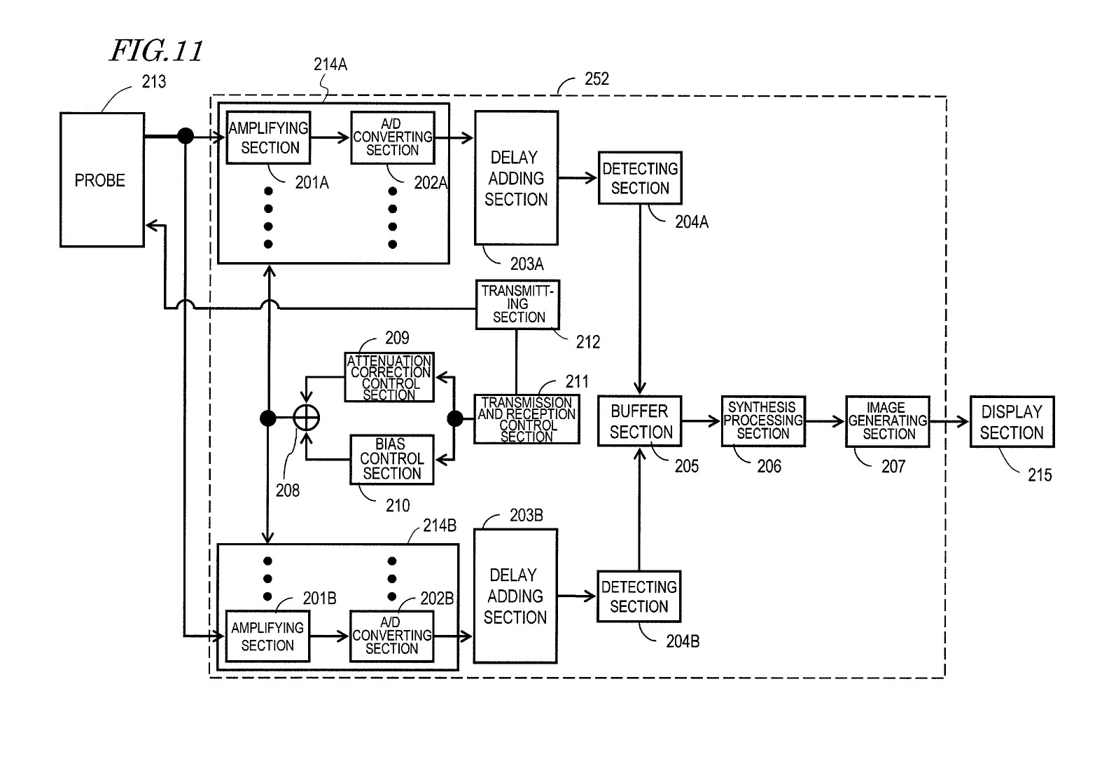

The synthesis processing section 206 includes a multiplier 206a, a switching section 206b and a switching decision section 206c. FIG. 9 is a flowchart showing how this synthesis processing section 206 operates. This synthesis processing is performed on the received signals on every acoustic line. The synthetic received signal may be generated on an acoustic line basis. Or when received signals for one frame are accumulated in the buffer section 205, synthetic received signals may be generated collectively for that one frame.

<Step 310>

The synthesis processing section 206 retrieves the first and second received signals that respectively have higher and lower reception sensitivities from the buffer section 205. The switching decision section 206c makes a threshold value decision by reference to the received signal with the higher reception sensitivity (corresponding to the higher amplification factor) that has been retrieved from the buffer section. Alternatively, the received signal to make reference to may also be the received signal with the lower reception sensitivity (corresponding to the lower amplification factor). In this case, the threshold value may be set arbitrarily. That is to say, the threshold value may be either a predetermined value that has been set in advance or determined dynamically and automatically based on a result of statistic analysis on the signal intensities of the received signals. For example, a histogram analysis may be carried out on the signal intensities and the center value of the histogram may be automatically determined to be the threshold value. If the intensity of the received signal is less than the threshold value, the process advances to Step 311. On the other hand, if the intensity of the received signal is equal to or greater than the threshold value, then the process advances to Step 312. The received signal, of which the intensity is smaller than the threshold value, falls within the segment with relatively low signal intensities. On the other hand, the received signal, of which the intensity is equal to or greater than the threshold value, falls within the segment with relatively high signal intensities.

<Step 311>

The switching decision section 206c turns the switching section 206b to output the received signal with the higher reception sensitivity (corresponding to the higher amplification factor).

<Step 312>

The switching decision section 206c turns the switching section 206b to output the received signal with the lower reception sensitivity (corresponding to the lower amplification factor). In this case, that received signal (corresponding to the lower amplification factor) is multiplied by a constant by the multiplier 206a.

As shown in FIG. 1, if the threshold value is determined by reference to the received signal with the higher reception sensitivity, the threshold value A2 may be used. On the other hand, if the threshold value is determined by reference to the received signal 601 with the lower reception sensitivity, the threshold value A1 may be used. As a result, the segments on the acoustic line can be sorted out into a segment with a fainter reflected echo and a segment with the stronger reflected echo. Consequently, a synthetic received signal can be generated by applying the portion 604 of the received signal 600 with the higher reception sensitivity to the segment 602 that corresponds to a region with the fainter reflected echo and that will define a dark region of a tomographic image and applying the portion 605 of the received signal 601 with the lower reception sensitivity to the segment 603 that corresponds to a region with the stronger reflected echo and that will define a bright region of the tomographic image.

The image generating section 207 generates a subject's tomographic image in either grayscales or color tones corresponding to the signal intensity of the synthetic received signal thus obtained. Consequently, a good tomographic image can be presented on the display section 215 in appropriate grayscales over the entire segment including dark and bright regions.

FIG. 10 schematically illustrates an image representing the blood vessel that was captured by the ultrasonic diagnostic apparatus 251. As shown in FIG. 10, the blood flow region 150 in which there is no strong reflector is displayed in multiple grayscales without causing underexposure, and the vascular wall 151 that is a strong reflector is also displayed in multiple grayscales without causing overexposure.

As described above, the ultrasonic diagnostic apparatus of this preferred embodiment synthesizes together two received signals with mutually different reception sensitivities and generates a tomographic image in grayscales or color tones corresponding to the signal intensity of the synthetic received signal thus obtained. Consequently, a good tomographic image can be displayed in appropriate grayscales or color tones over the entire segment including dark and bright regions.

### Embodiment 2

Hereinafter, a second preferred embodiment of an ultrasonic diagnostic apparatus according to the present invention will be described. FIG. 11 is a block diagram illustrating a configuration for an ultrasonic diagnostic apparatus 252. In FIG. 11, any component having substantially the same function as its counterpart of the first preferred embodiment described above is identified by the same reference numeral.

Unlike the first preferred embodiment described above, this ultrasonic diagnostic apparatus 252 includes two receiving sections 214A and 214B. Thus, there is no need to send ultrasonic waves twice in order to generate two received signals with mutually different reception sensitivities that have been amplified with different amplification factors. That is to say, two received signals with different reception sensitivities, which are needed to carry out high dynamic range imaging, can be obtained without decreasing the frame rate. On top of that, the bias of the amplification factor does not have to be controlled, either, every time an ultrasonic wave needs to be sent out.

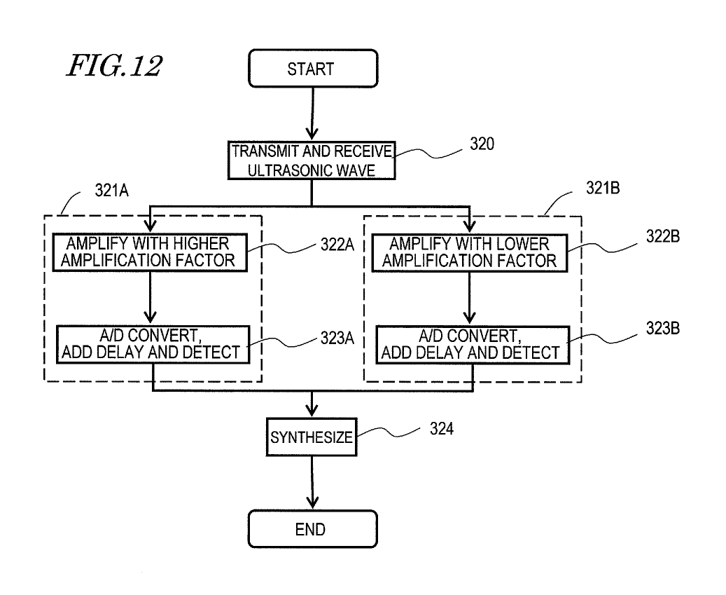

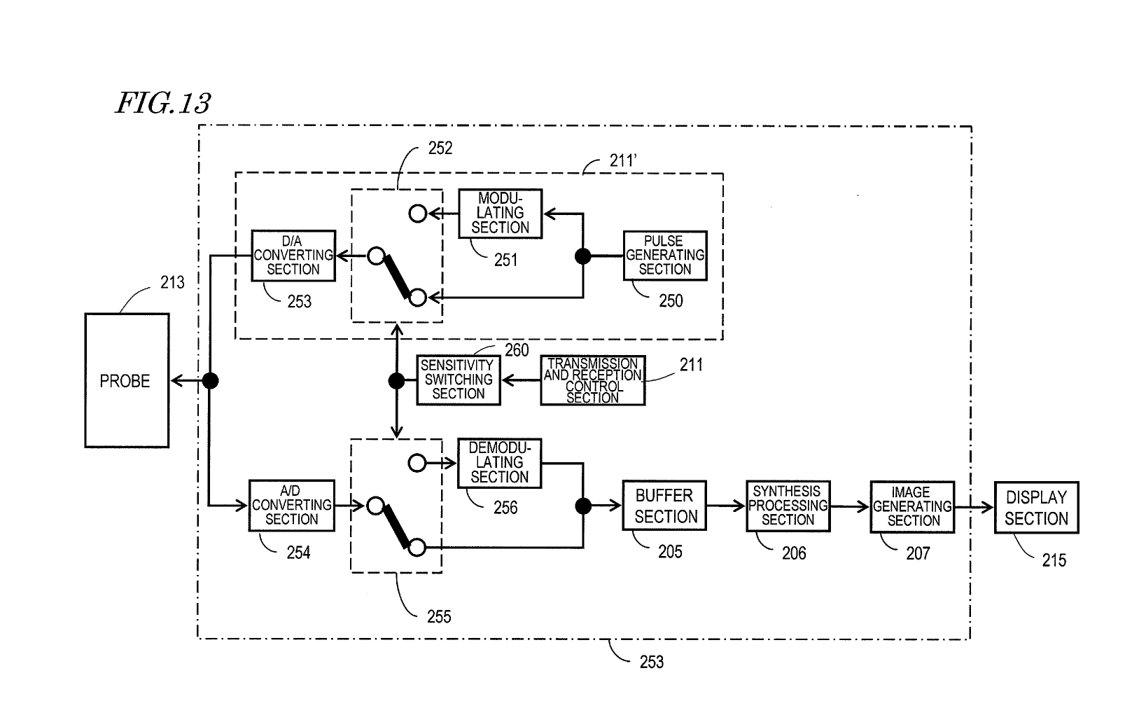

FIG. 12 is a flowchart illustrating how this ultrasonic diagnostic apparatus 252 works. Hereinafter, it will be described with reference to FIGS. 11 and 12 how this ultrasonic diagnostic apparatus 252 operates.

<Step 320>

An ultrasonic wave is transmitted and received. For that purpose, the probe 213 is driven by the transmitting section 212 to send out an ultrasonic wave toward the subject and then detects a reflected echo and generates a detection signal.

The operation of generating two received signals is performed as two series of steps 321A and 321B, which are carried out in parallel with each other.

<Steps 322A and 322B (to Be Performed in Parallel)>

Every time the transmission and reception control section 211 tells that it is time to send out an ultrasonic wave, the attenuation correction control section 209 determines an amplification factor for the purpose of attenuation correction control and the bias control section 210 determines two different amplification factors for the purpose of high dynamic range imaging. And those amplification factors are output to the adder 208. Then, each of the amplifying sections 201A and 201B amplify the detection signal with the sum of those amplification factors, thereby generating a received signal with the higher reception sensitivity (corresponding to the higher amplification factor) and a received signal with the lower reception sensitivity (corresponding to the lower amplification factor).

<Steps 323A and 323B (to Be Performed in Parallel)>

The A/D converting sections 202A and 202B respectively convert the received signal with the higher reception sensitivity (corresponding to the higher amplification factor) and the received signal with the lower reception sensitivity (corresponding to the lower amplification factor) into digital signals. Next, the delay adding sections 203A and 203B perform a focus control on the two received signals that have been converted into digital signals. Then, the detecting sections 204A and 204B carry out an envelope detection on the focus controlled received signals, and detected received signals get stored in the buffer section 205.

<Step 324>

As in the first preferred embodiment described above, the synthesis processing section 206 retrieves the data from the buffer section 205, and generates a synthetic received signal by the high dynamic range imaging method so that the received signal with the higher reception sensitivity is applied to a segment with the fainter reflected echo and the received signal with the lower reception sensitivity is applied to a segment with the stronger reflected echo on the same acoustic line. Then, the image generating section 207 generates the subject's tomographic image in either grayscales or color tones corresponding to the signal intensity of the synthetic received signal. As a result, the subject's tomographic image is presented on the display section 215.

According to this preferred embodiment, the amplification processing can be carried out in parallel with each other, and therefore, two received signals with mutually different reception sensitivities can be generated at the same time. Thus, there is no need to send ultrasonic waves twice on the same acoustic line and a signal with a relatively broad dynamic range can be generated without decreasing the frame rate.

### Embodiment 3

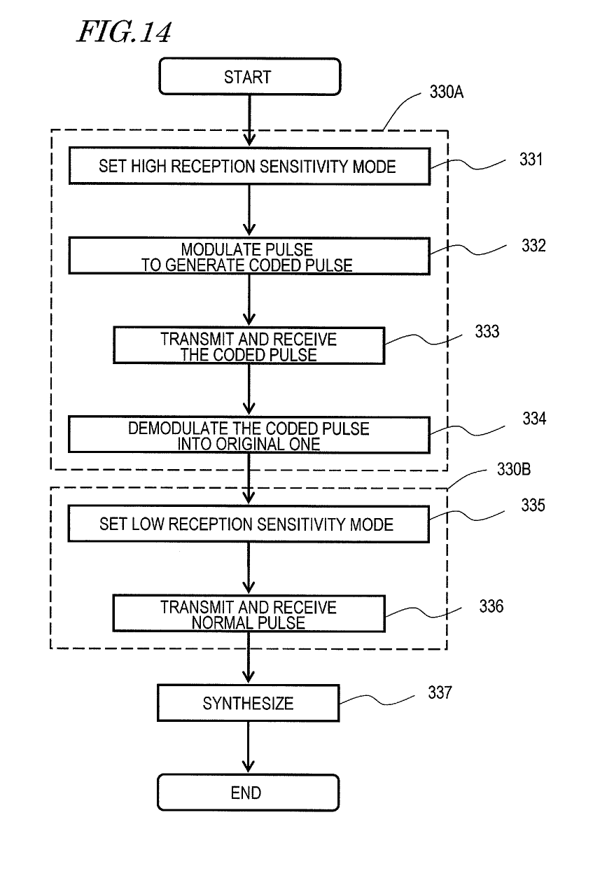

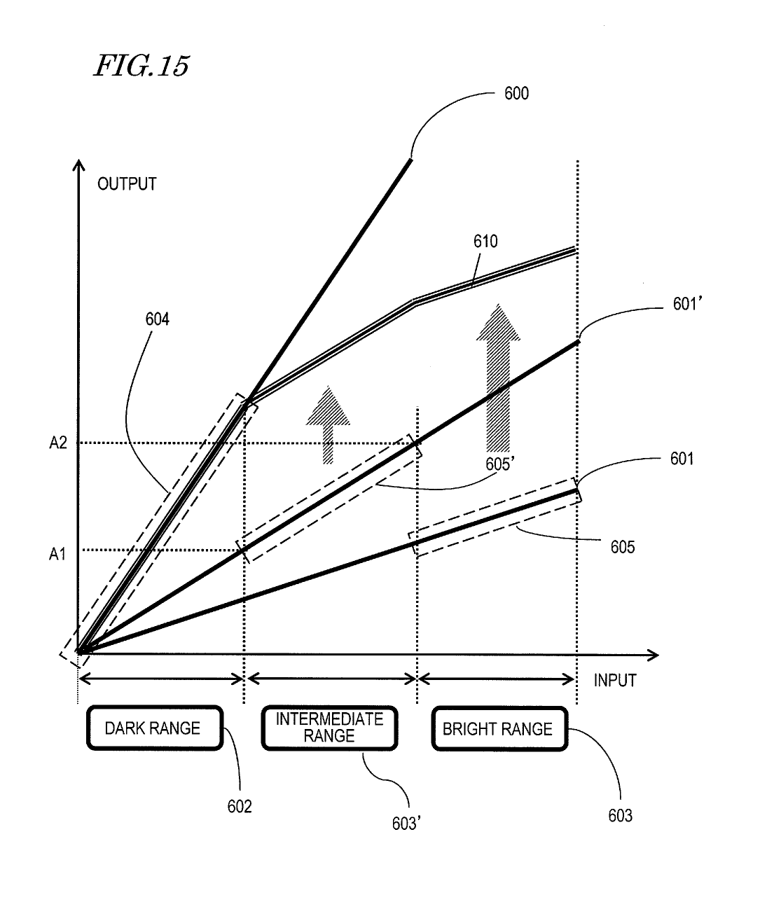

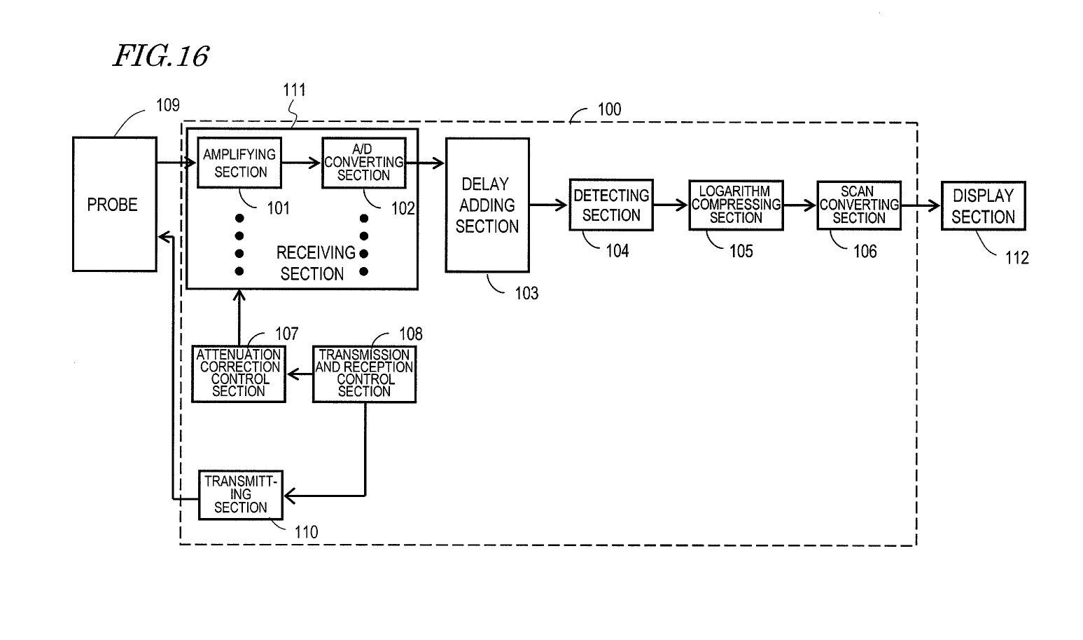

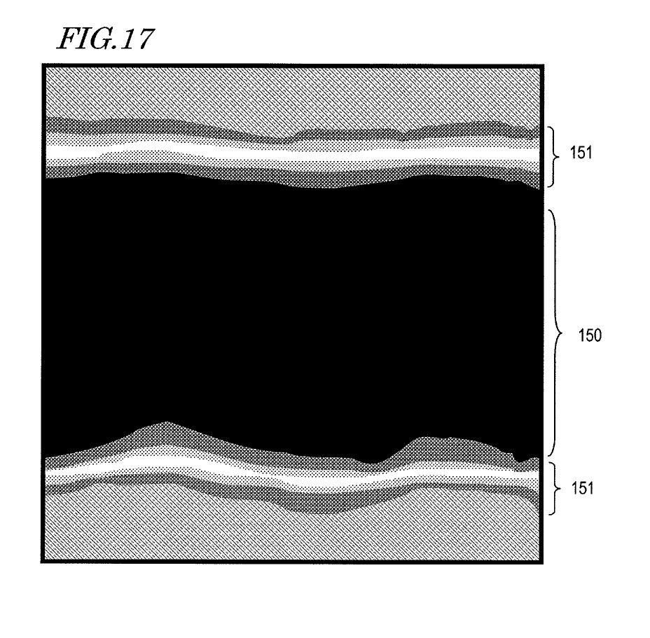

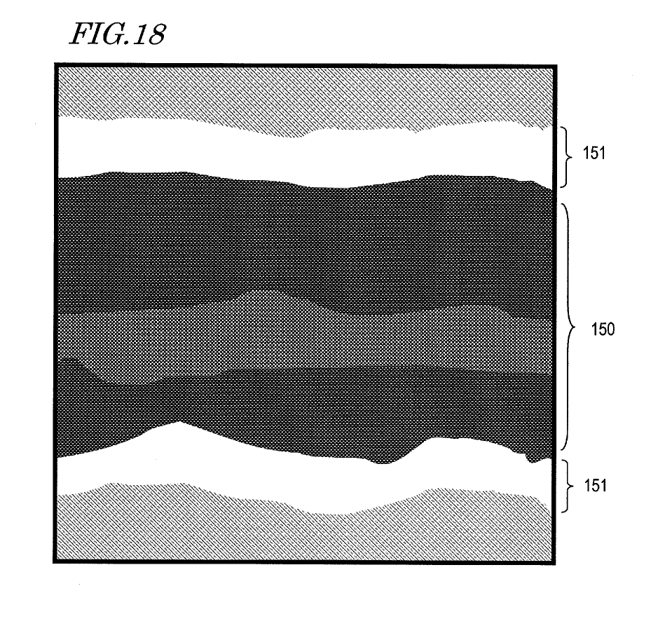

Hereinafter, a third preferred embodiment of an ultrasonic diagnostic apparatus according to the present invention will be described. FIG. 13 is a block diagram illustrating a configuration for an ultrasonic diagnostic apparatus 253. In FIG. 14, any component having substantially the same function as its counterpart of the first preferred embodiment described above is identified by the same reference numeral.

This ultrasonic diagnostic apparatus 253 transmits and receives an ultrasonic wave using a coded pulse signal to obtain a received signal with higher reception sensitivity and also transmits and receives an ultrasonic wave using a non-coded pulse signal to obtain a received signal with lower reception sensitivity.

For that purpose, the ultrasonic diagnostic apparatus 253 includes a transmitting section 211′, an A/D converting section 254, a switch 255, and a demodulating section 256. The transmitting section 211′ includes a pulse generating section 250, a modulating section 251, a switch 252 and a D/A converting section 253.

The pulse generating section 250 generates a digital pulse signal. The modulating section 251 performs code modulation on the pulse signal generated, thereby outputting a coded pulse signal. The switch 252 selects either the coded pulse signal or a non-coded pulse signal. The D/A converting section 253 converts either the coded pulse signal or non-coded pulse signal that has been selected by the switch 252 into an analog signal. As a result, the probe 213 is driven with the converted signal and sends out an ultrasonic wave toward the subject.

As in the first preferred embodiment described above, the detection signal obtained by the probe 213 is amplified by the amplifying section (not shown in FIG. 13), thereby generating a received signal. In this case, the amplifying section amplifies the detection signal with a constant amplification factor, no matter whether the ultrasonic wave received has been sent as the coded pulse signal or the non-coded pulse signal.

The A/D converting section 254 converts the received signal into a digital signal. As in the first preferred embodiment described above, the digitized received signal is subjected to a focus control by the delay adding section (not shown in FIG. 13). The switch 255 passes the focus controlled received signal to either the demodulating section 256 or the buffer section 205. In response to a signal supplied from a sensitivity switching section 260 in accordance with the instruction given by the transmission and reception control section 211, the switch 255 is turned so as to pass the received signal to the demodulating section 256 if the ultrasonic wave has been transmitted as a coded pulse signal. The demodulating section 256 demodulates the digitized detection signal and outputs the demodulated signal to the buffer section 205. Before being output to the buffer section 205, these received signals are subjected to envelope detection by the detecting section (not shown in FIG. 13) as in the first preferred embodiment described above.

The synthesis processing section 206 retrieves the data from the buffer section 205, and generates a synthetic received signal by the high dynamic range imaging method so that the received signal with the higher reception sensitivity is applied to a segment with the fainter reflected echo and the received signal with the lower reception sensitivity is applied to a segment with the stronger reflected echo on the same acoustic line. Then, the image generating section 207 generates the subject's tomographic image in either grayscales or color tones corresponding to the signal intensity of the synthetic received signal. As a result, the subject's tomographic image is presented on the display section 215.

Hereinafter, it will be described how this ultrasonic diagnostic apparatus 253 operates.

FIG. 14 is a flowchart showing how this ultrasonic diagnostic apparatus 253 works. In this case, a received signal with the higher reception sensitivity is generated by performing a series of steps 330A and a received signal with the lower reception sensitivity is generated by performing another series of steps 330B.

<Step 331>

First of all, a received signal with the higher reception sensitivity is generated. Every time the transmission and reception control section 211 tells the sensitivity switching section 260 that it is time to send out an ultrasonic wave, the sensitivity switching section 260 signals the switch 252 to connect the D/A converting section 253 to the modulating section 251 and also signals the switch 255 to connect the A/D converting section 254 to the demodulating section 256.

<Step 332>

Next, the pulse generating section 250 generates a digital signal pulse. In response, the modulating section 251 performs a code modulation on the pulse generated using a Barker code, for example, thereby generating a coded pulse signal.

<Steps 333 and 334>

The D/A converting section 253 converts the coded pulse signal into an analog signal. The probe 213 is driven with this signal and sends out an ultrasonic wave toward the subject. The echo reflected from the subject is detected by the probe 213 and the detection signal is amplified by the amplifying section (not shown in FIG. 13), thereby generating a received signal. The A/D converting section 254 converts the received signal into a digital signal and the demodulating section 256 decodes the received signal. Then, the decoded received signal is stored in the buffer section 205.

<Step 335>

Next, a received signal with the lower reception sensitivity is generated. Every time the transmission and reception control section 211 tells the sensitivity switching section 260 that it is time to send out an ultrasonic wave, the sensitivity switching section 260 signals the switch 252 to connect the D/A converting section 253 to the pulse generating section 250 and also signals the switch 255 to connect the A/D converting section 254 to the buffer section 205.

<Step 336>

An ultrasonic wave is transmitted and received on the same acoustic line as a non-coded pulse signal. The probe 213 detects the reflected echo and obtains a detection signal, on which a received signal is generated and stored in the buffer section 205.

<Step 337>

As already described in detail for the first preferred embodiment, the synthesis processing section 206 retrieves the data from the buffer section 205, and generates a synthetic received signal by the high dynamic range imaging method so that the received signal with the higher reception sensitivity is applied to a segment in which the detection signal has the lower signal intensity and the received signal with the lower reception sensitivity is applied to a segment in which the detection signal representing the ultrasonic wave received has the higher signal intensity on the same acoustic line.

According to this preferred embodiment, in order to generate a received signal with the higher reception sensitivity, an ultrasonic wave is transmitted and received using a coded pulse signal. Since the signal to drive the probe 213 has been coded, the signal is hardly affected by noise. And even if the reflected echo has low signal intensity, the reflected echo can still be detected with a high SNR. Consequently, even in a blood flow portion with low reflection intensity, the tomographic image causes no underexposure, and therefore, can be displayed in grayscales based on the difference in reflection intensity.

In the first through third preferred embodiments of the present invention described above, a synthetic received signal is supposed to be generated using two received signals with mutually different reception sensitivities. However, a synthetic received signal may also be generated using three or more received signals with mutually different reception sensitivities.

FIG. 15 illustrates an exemplary embodiment in which a synthetic received signal is generated using three received signals with mutually different reception sensitivities. If three received signals with mutually different reception sensitivities are used, the first preferred embodiment of the present invention described above is modified so that the transmitting section 212 transmits and receives ultrasonic waves and obtains detection signals three times on the same acoustic line. Then, the detection signals thus obtained are amplified with three different amplification factors, thereby obtaining a received signal 600 with relatively high reception sensitivity, a received signal 601 with relatively low reception sensitivity, and a received signal 601′ that has intermediate reception sensitivity between those of the two received signals 600 and 601.

The synthesis processing section 206 may classify the segments on the acoustic line according to the reflection intensity of the ultrasonic wave by applying the threshold values A1 and A2 to the received signal 601′. For example, the segments on the acoustic line may be sorted out by regarding a portion of the received signal 601′, of which the output is smaller than the threshold value A1, as a dark segment 602, another portion of the received signal 601′, of which the output is equal to or greater than the threshold value A1 but less than the threshold value A2, as an intermediate segment 603′, and still another portion of the received signal 601′, of which the output is equal to or greater than the threshold value A2, as a bright segment 603, respectively. By applying the portions 604, 605′ and 605 of the received signals 600, 601′ and 601 to the dark, intermediate and bright segments 602, 603′ and 603, respectively, and by synthesizing together the portions 605 and 605′, which have been multiplied by a constant or to which a constant has been added, and the portion 604, a synthetic received signal is generated.

By using a received signal that has been synthesized in this manner, a good tomographic image can be displayed in even more smoothly changing grayscales without causing any underexposure or overexposure. An embodiment in which three or more received signals with mutually different reception sensitivities are used has just been described as a modified example of the first preferred embodiment. However, a synthetic received signal can also be generated by using three or more received signals with mutually different reception sensitivities even in the second and third preferred embodiments described above.

Still alternatively, a synthetic received signal may also be generated using four or more received signals with mutually different reception sensitivities. Speaking more generically, if n received signals with mutually different reception sensitivities (where n is an integer that is equal to or greater than two) are used, the segments on each acoustic line are sorted out into n segments according to the reflection intensity of an ultrasonic wave. For example, the first segment may have the lowest reflection intensity and the n^{th} segment may have the highest reflection intensity. In that case, the n received signals with mutually different reception sensitivities get associated with n segments so that the higher the reflection intensity, the lower the reception sensitivity. Specifically, a received signal with the highest reception sensitivity (i.e., a received signal that has been amplified with the greatest amplification factor) gets associated with the first segment and a received signal with the lowest reception sensitivity (i.e., a received signal that has been amplified with the smallest amplification factor) gets associated with the n^{th} segment.

In the preferred embodiments described above, the image is supposed to be displayed in grayscales based on the signal intensity of the synthetic received signal. However, the image may also be displayed in color tones instead of grayscales. Optionally, the image may even be displayed with the grayscale and color tone both changed according to the signal intensity of the synthetic received signal.

### INDUSTRIAL APPLICABILITY

The ultrasonic diagnostic apparatus of the present invention can represent not only the blood flow but also the vascular wall in appropriate grayscales. For that reason, the present invention is applicable particularly effectively to making a diagnosis on the carotid artery, for example, when both the smoothness of the blood flow and the thickness of the vascular wall need to be inspected.

### REFERENCE SIGNS LIST

201, 201A, 201B amplifying section
202, 202A, 202B A/D converting section
203, 203A, 203B delay adding section
204, 204A, 204B detecting section
205 buffer section
206 synthesis processing section
207 image generating section
208 adder
209 attenuation correction control section
210 bias control section
211 transmission and reception control section
212 transmitting section
213 probe
214, 214A, 214B receiving section
215 display section

1. An ultrasonic diagnostic apparatus that sends out an ultrasonic wave toward a subject and receives the ultrasonic wave that has been reflected from the subject, thereby generating the subject's tomographic image, the apparatus comprising:
a receiving section for generating a number of received signals, which belong to the same acoustic line but which have mutually different reception sensitivities, based on a detection signal that has been obtained by detecting the ultrasonic reflected wave that has returned from the subject;
a synthesis processing section for classifying segments on the acoustic line according to the intensity of the ultrasonic reflected wave, and making a synthetic received signal out of the received signals based on a result of the classification; and
an image generating section for generating the subject's tomographic image by using either grayscales or color tones based on the signal intensity of the synthetic received signal.

2. The ultrasonic diagnostic apparatus of claim 1, wherein the received signals with the different reception sensitivities include a received signal with higher reception sensitivity and a received signal with lower reception sensitivity, and
wherein the synthesis processing section generates the synthetic received signal by applying the received signal with the higher reception sensitivity to a segment on the acoustic line where the ultrasonic wave has been reflected relatively faintly and the received signal with the lower reception sensitivity to a segment on the acoustic line where the ultrasonic wave has been reflected relatively strongly.

3. The ultrasonic diagnostic apparatus of claim 2, wherein the receiving section amplifies the detection signal with multiple different amplification factors, thereby generating the received signal with the higher reception sensitivity and the received signal with the lower reception sensitivity.

4. The ultrasonic diagnostic apparatus of claim 2, wherein the receiving section amplifies the ultrasonic wave detection signal with a higher amplification factor and with a lower amplification factor in parallel with each other, thereby generating the received signal with the higher reception sensitivity and the received signal with the lower reception sensitivity.

5. The ultrasonic diagnostic apparatus of claim 2, further comprising a transmitting section for generating a pulse signal and a coded pulse signal and driving a probe that sends out the ultrasonic wave,
wherein the receiving section generates the received signal with the higher reception sensitivity by making the probe detect and demodulate the ultrasonic wave that has been sent out as the coded pulse signal and also generates the received signal with the lower reception sensitivity by making the probe receive the ultrasonic wave that has been sent out as the pulse signal.

6. The ultrasonic diagnostic apparatus of claim 2, wherein the receiving section amplifies the detection signal with the amplification factor increased with time while the ultrasonic wave is being transmitted with respect to the same acoustic line.

7. The ultrasonic diagnostic apparatus of claim 2, wherein based on a result of statistical analysis on the signal intensities of the detection signal, the synthesis processing section automatically determines a segment with the lower signal intensity and a segment with the higher signal intensity.

8. The ultrasonic diagnostic apparatus of claim 1, further comprising a transmitting section for driving a probe that sends out the ultrasonic wave,
wherein the transmitting section drives and instructs the probe to scan the subject with ultrasonic waves that have been sent out n times (where n is an integer that is equal to or greater than two) per acoustic line, and
wherein the receiving section amplifies the detection signal that has been obtained by the probe with the higher amplification factor and with the lower amplification factor alternately.
